(19) United States
(12) Patent Application Publication
(10) Pub. No.: US 2016/0136058 A1
(43) Pub. Date: May 19, 2016

(54) SYSTEM FOR FILLING A ROOT CANAL OF A TOOTH AND FOR COVERING PULP

(71) Applicant: COLTÈNE/WHALEDENT AG, Altstaetten (CH)

(72) Inventors: Martin SCHLÜTER, Wangen (DE); Benjamin EZEH, Rankweil (AU); Werner MANNSCHEDEL, Langenau (DE)

(21) Appl. No.: 14/901,502

(22) PCT Filed: Jun. 18, 2014
(86) PCT No.: PCT/EP2014/062875

§ 371 Date: Dec. 28, 2015

(30) Foreign Application Priority Data
Jul. 10, 2013 (EP) 13175925.0

(51) Int. Cl. A61K 6/00

(52) U.S. Cl. CPC A61K 6/0038; A61K 6/0008; A61K 6/0073; A61K 6/083

(57) ABSTRACT

A system for filling a root canal of a tooth and for covering pulp, with a silicone material comprising MₓO_{y}, where MₓO_{y} is selected from the group comprising CaO, BaO, MgO, Na₂O, K₂O, and SrO, especially preferably CaO; and/or at least one compound containing MₓO_{y}, where MₓO_{y} is selected from the group comprising CaO, BaO, MgO, Na₂O, K₂O, and SrO, especially preferably a compound containing CaO, in particular a compound containing CaO/SiO₂. Such compositions exhibit bioactive properties and improve the ease of use and the prognosis for success of the system, according to the present invention, for filling a root canal and for covering pulp.

The invention relates to the field for the care of root canals in dentistry, in particular compositions for the filling of root canals and for the covering of pulp.

In the conventional treatment of irreversible damage to the dental pulp, the diseased pulp is mechanically removed from the root canal and the root canal is cleaned and drilled out, filled with an elastic-plastic element or another filling material and afterwards sealed. An ideal root canal filling material should not irritate the periapical tissue, tightly close off the root canals laterally and vertically, be stable in volume and not at any rate shrink in the root channel. Reference may be made, for the state of the art, for example, to Friedman et al. in J. Dent. Res., 54 1975, 921-925, and Briseno in Philipp J., 7(2) 1990, 65-73 and U.S. Pat. No. 4,632,977. Briseno describes, as root canal filling materials, inter alia, semisolid cements based on artificial resin, on zinc oxide-eugenol, on calcium hydroxide and on glass ionomer. U.S. Pat. No. 4,632,977 proposes filling materials based on trans-polyisoprene, for example based on gutta-percha or balata. Such compositions, for example as gutta-percha molding articles, or “gutta-percha points”, are available commercially. They usually comprise approximately 20% by weight of gutta-percha matrix, 60 to 75% by weight of zinc oxide as filler, i to 17% by weight of heavy metal sulfates as X-ray contrast medium and 3 to 4% by weight of waxes and resins as plasticizer. Such gutta-percha compositions are thermoplastic and can accordingly be satisfactorily attached in the flowable state on the inside of the root canal.

EP 864 312 teaches a system for the filling of root canals of teeth which is based on an addition-crosslinking silicone composition. A polyisoprene-based (for example gutta-percha) root canal post can additionally be embedded in such a composition. However, the ease of use of such a system is, however, not optimal under all conditions.

EP 951 895 teaches a system in which a gutta-percha powder is directly incorporated in a sealer (for example based on silicone materials). The ease of use and the reproducibility of the results of the system of sealer and gutta-percha could already be improved through this.

Likewise, the application of inorganic trioxide aggregate in the prepared root point, in which the root canal was subsequently closed up conventionally with gutta-percha and sealer, has been described (Schweiz. Monatsschr. Zahnmed., Vol. 114: 3/2004, pp. 223-230). However, the laborious handling of several materials in several stages, and also the inconvenience for the patients associated therewith due to the several visits necessary, are disadvantageous in this connection.

It is an object of the invention to further improve the ease of use and the treatment success of systems for the filling of the root canal.

This object is achieved through a system for filling a root canal of a tooth based on a silicone material, comprising
MₓO_{y}, in which MₓO_{y} is chosen from the group consisting of CaO, BaO, MgO, Na₂O, K₂O and SrO, particularly preferably CaO;
and/or
at least one MₓO_{y}-comprising compound, in which MₓO_{y} is chosen from the group consisting of CaO, BaO, MgO, Na₂O, KO and SrO, particularly preferably a CaO-comprising compound, in particular a CaO/SiO₂-comprising compound;
   and also mixtures thereof.

The term “system” is understood to mean, in the context of the invention, both one- and two-component presentation forms of the root-filling compositions. In a one-component composition, the crosslinking constituents for the silicone composition are already present premixed; the crosslinking is then carried out via the admission of moisture. In two-component systems, the constituents of a composition capable of the crosslinking reaction are present separately but arranged for use together. The crosslinking of such two-component systems is generally carried out independently of the admission of moisture.

The abovementioned systems of the state of the art based on silicone materials are inert; neither do they support or accelerate in particular the renewal of hard structure. It has now been found that the additional presence of MₓO_{y}, the latter being chosen from the group consisting of CaO, BaO, MgO, Na₂O, K₂O and SrO, or of an MₓO_{y}-comprising compound (in particular of a CaO/SiO₂-comprising compound) in a silicone material significantly promotes the formation of hard structure. The percentage by weight of the MₓO_{y} (in particular of the CaO) or of the abovementioned MₓO_{y}-comprising compounds preferably lies in the range from 3% by weight to 80% by weight. That this moreover does not counteract the processability and the resulting sealing properties of the silicone material per se could likewise not be expected. Contrary to expectation, this formation of hard structure has a positive effect on the sealing properties. In particular, cracks and weak points in the dentin can be better compensated for. Moreover, undesirable, for example brown, discolorations can be avoided.

In addition, a surprisingly very reliable and durable pulp covering can be achieved with the system according to the invention.

In preferred embodiments, the MₓO_{y} (which is chosen from the group consisting of CaO, BaO, MgO, Na₂O, K₂O and SrO, particularly preferably CaO) or the MₓO_{y}-comprising compound exhibits a mean particle size (weighted average) of <100 μm, preferably in the range from 0.01 to 90 μm, more preferably in the range from 0.1 to 90 μm and particularly preferably in the range from 1 to 60 μm. With such particle sizes, the MₓO_{y} (in particular the CaO) or the MₓO_{y}-comprising compound can be very satisfactorily incorporated in the silicone composition, and the effects described above of the renewal of hard structure are also well expressed.

Particularly preferably, the system further exhibits at least one isoprene-based polymer. The isoprene-based polymer can in this connection be chosen from the group consisting of polyisoprene, in particular trans-1,4-polyisoprene, gutta-percha, balata and also mixtures thereof. The isoprene-based polymer in this case exhibits a mean particle size (weighted average) of <100 μm, in particular in the range from 1 to 60 μm, preferably from 2 to 45 μm, more preferably from 5 to 30 μm and particularly preferably from 10 to 30 μm. It has been found, with such compositions, for example based on addition crosslinking silicones and gutta-percha powder, that they still exhibit the advantageous properties of the compositions of EP 951 895 but in addition the formation of hard structure is promoted, as described above.

Particularly preferred alternative forms of the invention concern addition-crosslinking silicone systems with components A and B present separately, in which
the component A comprises at least one or more silicone oil(s) with at least two Si—H groups or consists of these;
the component B comprises at least one or more silicone oil(s) with at least two vinyl groups or consists of these.

Optionally, at least one of the components A or B can comprise a catalyst for the addition reaction of Si—H groups with vinyl groups.

The crosslinking of the silicone material is carried out in these embodiments of the invention via a hydrosilylation reaction, schematically as follows:

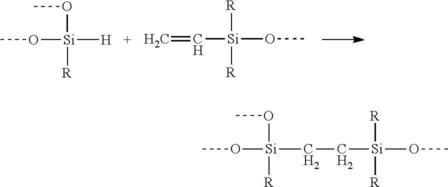

R describes, in this connection, any alkyl moiety, which can be identical or different. In particularly preferred embodiments of the invention, R describes a methyl group; these systems are thus based on methylhydrosilanes and dimethylsiloxanes,

The platinum catalysts known per se are preferably used as catalysts for hydrosilylation reactions.

More preferably, a slight expansion of the silicone material can be provided for, for example through a controlled swelling or through a partial dehydrogenating coupling, schematically as follows:

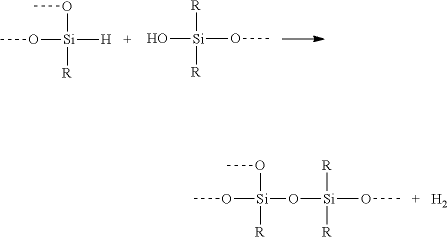

For its part, R describes, in this connection, any alkyl moiety, which can be identical or different (embodiments with R═CH₃ are, however, preferred). The emergence of hydrogen results in foam formation in the crosslinking of the silicone material, which foam formation can be easily adjusted, via the content of silicone oils comprising hydroxyl groups, to a desired amount by a person skilled in the art using routine tests.

Optionally, a catalyst known per se for this can be used as catalyst for dehydrogenating coupling, such as, for example, platinum catalysts, zinc octoate, iron octoate, dibutyltin dilaurate or a compound of the general formula Sn(OOCR)₂, in which R describes an alkyl moiety.

In all two-component systems described here, it is the case that
the at least one isoprene-based polymer; and/or
MₓO_{y}, which is chosen from the group consisting of CaO, BaO, MgO, Na₂O, K₂O and SrO, particularly preferably CaO; and/or
at least one MₓO_{y}-comprising compound, in which MₓO_{y} is chosen from the group consisting of CaO, BaO, MgO, Na₂O, K₂O and SrO, particularly preferably a CaO-comprising compound, in particular a CaO/SiO₂-comprising compound;
   can be present both in component A and in component B. Very particularly preferably, the mentioned constituents, insofar as they are present, are uniformly distributed over the two components A and B. It is possible, through this, to achieve a particularly balanced distribution of these constituents in the finished mixture, without expensive mixers being necessary.

Alternatively, the system according to the invention can also be formed by condensation-crosslinking. It then typically comprises:
i) at least one or more silicone oil(s) with at least two Si—OH groups;
ii) at least one or more silicone oil(s) with at least two functional groups which are capable of reacting with Si—OH groups, optionally with admission of moisture.

In these embodiments, the silicone oils can be made available as a one- or two-component system. The condensation-crosslinking systems of most practical importance comprise, as constituent ii), silicone oils with functional silane end groups, such as, for example:

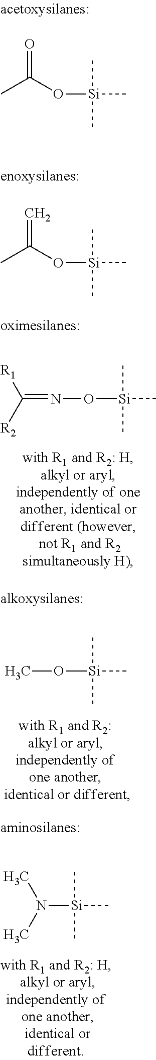

Optionally, a catalyst known per se can be used as catalyst for condensation-crosslinking, such as, for example, zinc octoate, iron octoate, dibutyltin dilaurate or a compound of the general formula Sn(OOCR)₂, in which R describes an alkyl moiety.

In the context of the invention, the presence of Al₂O₃ (and the Fe₂O₃ frequently occurring together with it) can be dispensed with. In view of some studies on the supposed neurotoxicity of aluminum, Al₂O₃-free compositions are particularly preferred in the context of the invention. It has been surprisingly shown that the presence of Al₂O₃ (in particular of C₃A (tricalcium aluminate, (CaO)₃.Al₂O₃), C₂AF (dicalcium aluminoferrite, (CaO)₂.Al₂O₃.Fe₂O₃) and C₄AF (tetracalcium aluminoferrite, (CaO)₄.Al₂O₃.Fe₂O₃), which are all typical constituents of MTA) is not crucial for the operation of the invention.

The presence of CaSO₄.2H₂O (gypsum) is likewise possible in the context of the invention but not necessary.

The effects of the invention are not yet completely understood. Without being committed to this explanation, it is, however, at the moment assumed that, surprisingly, the silicone materials of these compounds are not fully capable of moistening, so that these materials are still at least partly exposed on the surface of the silicone material in such a way that they, under the influence of body fluid, cause the formation of hard structure (in particular hydroxyapatite).

Apatite is the abbreviated and generic name for a group of chemically similar but not closely defined minerals which are known to a person skilled in the art. It also appears possible that the apatite formation takes place via leeching of Ca²⁺, Ba²⁺, Mg²⁺, Sr², Na⁺ or K⁺ out of the composition, which ions are replaced by H₃O⁺ ions on the surface of the composition. The SiOH groups on the surface of the composition produced through this could induce the formation of apatite seed crystals which, through the raising of the ion activity product (IAP), could be yet strengthened. Seed crystals, once formed, can spontaneously grow further because of the calcium and phosphate ions present in excess in the body fluid. The body fluid is simulated in the laboratory; reference is made to “simulated body fluid”, SBF (Kokubo et al., J. Biomed. Mater. Res., 24 (1990), 721-734). The ion concentrations of SBF used here and subsequently are as follows (in comparison with human blood plasma), the pH being adjusted to 7.25 at 36.5° C. using 50 mM tris(hydroxymethyl)aminomethane and 45 mM HCl:

Concentration [mmol/dm³]

| | SBF | Blood plasma |
|---|---|---|
| Na⁺ | 142.0 | 142.0 |
| K⁺ | 5.0 | 5.0 |
| Mg²⁺ | 1.5 | 1.5 |
| Ca²⁺ | 2.5 | 2.5 |
| Cl⁻ | 147.8 | 103.0 |
| HCO₃⁻ | 4.2 | 27.0 |
| HPO₄²⁻ | 1.0 | 1.0 |
| SO₄²⁻ | 0.5 | 0.5 |

The production of hydroxyapatite from, for example, calcium phosphate and calcium hydroxide is admittedly known per se (cf. EP 367 808) but under strongly basic conditions (pH>11), summarizing as follows:

3Ca(H₂PO₄)₂.2H₂O+7Ca(OH)₂→2Ca(PO₄)₃OH+18H₂O.

However, such strongly basic reaction conditions are obviously undesirable in vivo. Moreover, it has been shown, in the context of the invention, that the formation of hydroxyapatite takes places astonishingly quickly and under mild conditions in SBF, in particular SBF with a pH of 7.25, even starting from CaO or SiO₂/CaO.

In additional preferred embodiments, the system according to the invention can comprise the at least one CaO/SiO₂-comprising compound(s) in the form of a glass and/or a glass-ceramic. Such a glass can be a binary CaO/SiO₂ composition, a ternary CaO/P₂O₅/SiO₂ composition or also a quaternary SiO₂/CaO/P₂O₅/Na₂O composition; mixtures of the abovementioned compositions are obviously likewise possible.

In the context of the invention, preferred glasses are known and available under the Bioglass® brand (and also, for example, from Schott as “bioactive glass”); see, regarding this, Hench in J. Mater. Sci: Mater. Med., (2006) 17, 967-978 (the disclosure of this document with regard to the Bioglass® compositions is herewith included in this document by way of reference). In the context of the invention, suitable bioactive glasses are further described in Hupa, p. 3 ff., in Bioactive glasses: Materials, properties and applications, 2011, Woodhead Publishing Ltd., ISBN 1845697685 (the disclosure of this book chapter is, considering the composition of bioactive glasses, herewith incorporated in this document by way of reference).

The glass can particularly preferably, in the context of the invention, be chosen from the glasses i) and ii), which exhibit the following constituents (besides P₂O₅ with an amount typically between 2 and 6% by weight, in individual cases up to approximately 20% by weight):
i) from 35 to 65 mol %, in particular from 35 to 60 mol %, SiO₂,
   from 10 to 50 mol % CaO,
   from 5 to 40 mol % Na₂O;
ii) from 50.01 to 65 mol % SiO₂,
   from 1 to 9.99 mol % CaO,
   from 5 to 40 mol % Na₂O.

The glass is very particularly preferably chosen from the group consisting of 45S5, 58S, S70C30, S53P4 and mixtures thereof.

All abovementioned systems preferably also comprise an X-ray contrast medium, in particular chosen from compounds of the group consisting of zinc, ytterbium, yttrium, gadolinium, zirconium, strontium, tungsten, tantalum, niobium, barium, bismuth, molybdenum, lanthanum; alloys, fluorides, sulfates, carbonates, tungstates, carbides and oxides all abovementioned elements, particularly preferably YbF₃ or ZrO; organic and inorganic iodine compounds. The respective constituent amounts in which the X-ray contrast medium is to be added to the composition in order to obtain the desired contrast can be easily determined by a person skilled in the art using routine tests.

The systems according to the invention as described above can be made available preproportioned in one or more capsule(s), cartridge(s) (in particular double-chamber syringes) or tubular bag(s). This applies both for one- and two-component systems. In two-component systems, the components A and B can particularly preferably be made available separately from one another in a joint capsule, cartridge (in particular a double-chamber syringe) or tubular bag, it being possible for the capsule to be inserted into a dispenser. The handling is simplified in such a presentation form in a way known per se. The provision of the components A and B in separate capsules, cartridges (in particular double-chamber syringes) or tubular bags is, however, obviously likewise possible.

Systems according to the invention as described above can further comprise at least one root canal post, in particular based on an isoprene polymer. These root canal posts can, in a way known per se, be encapsulated with the compositions according to the invention in a root canal of a tooth.

Additional possible constituents of the system according to the invention as described above are dispensers for capsule(s), cartridge(s) (in particular double-chamber syringe(s)) or tubular bag(s); applicator tips for flowable compositions (for application of the compositions according to the invention based on a silicone material); and also marking aid movable on an applicator tip for determining the root canal depth. Such a marking aid can, for example, be an elastic ring which can be rolled up and unrolled on the applicator tip.

The invention is explained below using exemplary embodiments and figures, without the subject matter of the invention being limited to these embodiments. In this connection:

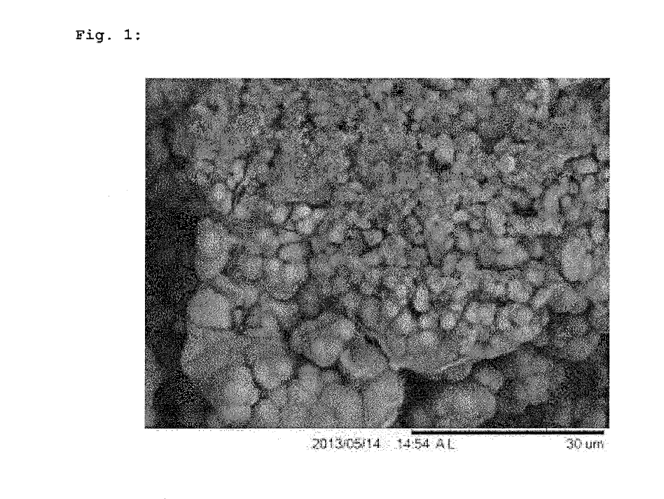

FIG. 1 shows an SEM photograph of a silicone pellet comprising a CaO/SiO₂ mixture, 14 d after imbibing in an SBF buffer;

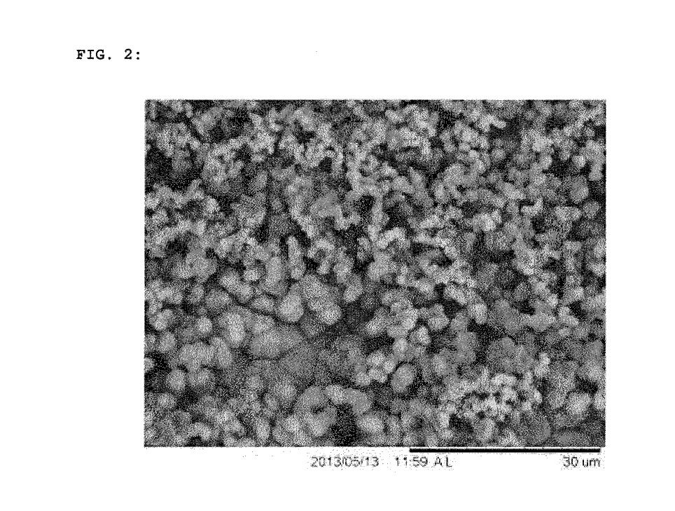

FIG. 2 shows an SEM photograph of the silicone pellet comprising bioactive glass, 14 d after imbibing in an SBF buffer.

### BIOACTIVITY OF A CAO/SIO₂ MIXTURE IN SILICONE MATERIAL

The following mixture was prepared:

| | % by | % by | |
|---|---|---|---|
| | weight | weight | 4:1 |
| | Base | Cat | mixture |
| Divinylpolydimethyl- | 26.35 | 34.23 | 27.93 |
| siloxanes | | | |
| Hydromethylpolydivinyl- | 9.25 | | 7.40 |
| siloxanes | | | |
| Silicates | 3.67 | 5.76 | 4.09 |
| X-ray opaque materials | 14.00 | 60.00 | 23.20 |
| Pigments | 1.72 | | 1.38 |
| Gutta-percha mixture | 20.00 | | 16.00 |
| Pt catalyst | | 0.01 | |
| CaO/SiO₂ mixture* | 25.00 | | 20.00 |
| | 100.00 | 100.00 | 100.00 |

*A mixture of CaO (65% by weight), SiO₂ (25% by weight) and Ca(HPO₄)₂ (10% by weight) was used as CaO/SiO₂ mixture.

Test specimens in the form of pellets were prepared from the paste obtained. For this, a plastic film was spread out in a split ring mold with an internal diameter of 20 mm and a height of 1.5 mm, and a piece of dental floss was placed thereon. After that, the split ring mold was filled with the mixture described above and a second plastic film was applied and weighed down with a sheet of glass. After three hours, the cured pellets were thereupon removed from the split ring mold. The pellets consequently obtained were then imbibed on the dental floss hanging at 37° C. in an SBF buffer with a pH of 7.25.

An SEM photograph after imbibing for 14 days in the SBF buffer can be seen in FIG. 1. Before taking the SEM photograph, the pellet was each time rinsed with ultrapure water and subsequently dried. It is obvious that crystals have been formed on the surface of the pellets. The spherical crystals have the form, known to a person skilled in the art, of apatite crystals. EDX measurements and also IR and Raman spectroscopy have likewise confirmed the formation of apatite (data not shown).

Bioactivity of Glass Materials in Silicone Material

The following mixture was prepared:

| | % by | % by | |
|---|---|---|---|
| | weight | weight | 4:1 |
| | Base | Cat | mixture |
| Divinylpolydimethyl- | 26.35 | 34.23 | 27.93 |
| siloxanes | | | |
| Hydromethylpolydivinyl- | 9.25 | | 7.40 |
| siloxanes | | | |
| Silicates | 3.67 | 5.76 | 4.09 |
| X-ray opaque materials | 14.00 | 60.00 | 23.20 |
| Pigments | 1.72 | | 1.38 |
| Gutta-percha mixture | 20.00 | | 16.00 |
| Pt catalyst | | 0.01 | |
| Bioactive glass** | 25.00 | | 20.00 |
| | 100.00 | 100.00 | 100.00 |

**G018-144 (Schott AG) was used as bioactive glass.

Test specimens in the form of pellets were prepared from the paste obtained. For this, a plastic film was spread out in a split ring mold with an internal diameter of 20 mm and a height of 1.5 mm, and a piece of dental floss was placed thereon. After that, the split ring mold was filled with the mixture described above and a second plastic film was applied and weighed down with a sheet of glass. After three hours, the cured pellets were thereupon removed from the split ring mold. The pellets consequently obtained were then imbibed on the dental floss hanging at 37° C. in an SBF buffer with a pH of 7.25.

An SEM photograph after imbibing for 14 days in the SBF buffer can be seen in FIG. 2. Before taking the SEM photograph, the pellet was each time rinsed with ultrapure water and subsequently dried. It is obvious that crystals have been formed on the surface of the pellets. The spherical crystals have the form, known to a person skilled in the art, of apatite crystals. EDX measurements and also IR and Raman spectroscopy have likewise confirmed the formation of apatite (data not shown).

1-13. (canceled)

14. A system for filling a root canal of a tooth and for covering pulp based on a silicone material, the system comprising
MₓO_{y}, in which MₓO_{y} is selected from the group consisting of CaO, BaO, MgO, Na₂O, K₂O and SrO;
and/or
at least one MₓO_{y}-comprising compound, in which MₓO_{y} is selected from the group consisting of CaO, BaO, MgO, Na₂O, K₂O and SrO;
and also mixtures thereof.

15. The system according to claim 14, wherein said MₓO_{y} and/or said at least one MₓO_{y}-comprising compound exhibits or exhibit a mean particle size (weighted average) of <100 μm.

16. The system according to claim 14, wherein the system comprises at least one isoprene-based polymer.

17. The system according to claim 16, wherein said isoprene-based polymer is selected from the group consisting of polyisoprene, gutta-percha, balata and mixtures thereof.

18. The system according to claim 16, wherein said isoprene-based polymer exhibits a mean particle size (weighted average) of <100 μm.

19. The system according to claim 14, comprising component A and component B present separately, in which
said component A comprises at least one or more silicone oil(s) with at least two Si—H groups or consists of these;
said component B comprises at least one or more silicone oil(s) with at least two vinyl groups or consists of these.

20. The system according to claim 19, wherein at least one of the components A or B comprises a catalyst for an addition reaction of the Si—H groups with the vinyl groups.

21. The system according to claim 19, wherein
said at least one isoprene-based polymer; and/or
MₓO_{y}, which is selected from the group consisting of CaO, BaO, MgO, Na₂O, K₂O and SrO; and/or
at least one MₓO_{y}-comprising compound, in which MₓO_{y} is selected from the group consisting of CaO, BaO, MgO, Na₂O, K₂O and SrO, and
is/are present both in component A and in component B.

22. The system according to claim 14, wherein the system comprises:
i) at least one or more silicone oil(s) with at least two Si—OH groups;
ii) at least one or more silicone oil(s) with at least two functional groups which are capable of reacting with the Si—OH groups; and
said silicone oils are made available as a one- or two-component system.

23. The system according to claim 14, wherein the at least one MₓO_{y} comprising compound is a CaO/SiO₂-comprising compound, at least one of the CaO/SiO₂-comprising compound(s) is a glass and/or a glass-ceramic, the glass is selected from the group consisting of binary CaO/SiO₂ compositions, ternary CaO/P₂O/SiO₂ compositions and quaternary SiO₂/CaO/P₂O₅/Na₂O compositions.

24. The system according to claim 14, wherein said silicone material comprises an X-ray contrast medium selected from compounds of the group consisting of zinc, ytterbium, yttrium, gadolinium, zirconium, strontium, tungsten, tantalum, niobium, barium, bismuth, molybdenum, lanthanum; alloys, fluorides, sulfates, carbonates, tungstates, carbides and oxides of all abovementioned elements; organic and inorganic iodine compounds.

25. The system according to claim 14, preproportioned in one or more capsule(s), cartridge(s), double-chamber syringe(s) or tubular bag(s).

26. The system according to claim 14, further comprising at least one root canal post, based on an isoprene polymer.

27. The system according to claim 14, further comprising one or more constituents selected from the group consisting of dispensers for capsule(s), cartridge(s), double-chamber syringes or tubular bag(s); applicator tips for flowable compositions; and marking aids movable on an applicator tip for determining the root canal depth.
